(19) United States
(12) Patent Application Publication
(10) Pub. No.: US 2026/0218258 A1
(43) Pub. Date: Jul. 30, 2026

(54) PIGMENT PREPARED BY USING MICROORGANISMS AND METHOD FOR PREPARING SAME

(71) Applicants: COSMAX, INC., Gyeonggi-do (KR); COSMAX AB Inc., Seoul (KR)

(72) Inventors: Ju Yun KIM, Seoul (KR); Ju Hyun SON, Seoul (KR); Kwang Nyeon KIM, Seoul (KR)

(21) Appl. No.: 19/137,554

(22) PCT Filed: Dec. 20, 2023
(86) PCT No.: PCT/KR2023/021146

§ 371 Date: Jun. 10, 2025

(30) Foreign Application Priority Data
Dec. 20, 2022 (KR) 10-2022-0179001

(51) Int. Cl. C12P 17/16; C09B 61/00

(52) U.S. Cl. CPC C12P 17/165; C09B 61/00

(57) ABSTRACT

The present invention relates to a pigment extract prepared by using microorganisms and a method for preparing same. According to the present invention, there is provided a pigment derived from natural substances as an active ingredient having functions such as killing bacteria and healing wounds. The pigment of the present invention can be utilized in various modifications by using substitution of metal ions or functional groups.

### TECHNICAL FIELD

The present invention relates to a pigment extract prepared by using skin flora and a method for preparing the same. This application claims the benefit of priority from Korean Patent Application No. 10-2022-0179001, filed Dec. 20, 2022, the entire contents of which are incorporated herein by reference.

### BACKGROUND ART

The human body can be a habitat for various microorganisms, and microorganisms form a symbiotic relationship with the host and affect the host. Among these, skin flora present on the skin are microorganisms that live on the surface of the skin and are mainly found in the outermost layer of the epidermis and the upper part (upper layer) of the hair follicles. There are approximately 1,000 species of microorganisms as skin flora. Among the microorganisms present on the skin, aerobic microorganisms can secrete lipolytic enzymes that can utilize lipids in the epidermal layer.

Meanwhile, tar pigments and synthetic pigments are generally used as pigments for cosmetics. However, these types of pigments may be absorbed into the body through the skin and are difficult to excrete, which can cause toxicity and may cause skin irritation, allergies, or carcinogenesis.

Therefore, there is a need to manufacture pigments using human-friendly raw materials and methods.

### DETAILED DESCRIPTION OF THE INVENTION

### Technical Problem

The purpose of the present invention is to provide a pigment that can be applied to the skin and is harmless to the human body by using skin flora.

### Solution to Problem

In order to solve the above problem, the present invention provides a pigment produced by a microorganism of skin flora with the accession number KCTC15741BP (Korea Research Institute of Bioscience and Biotechnology, Biological Resource Center). Specifically, the pigment may include a porphyrinoid-based substance.

According to another embodiment of the present invention, there is provided a method for preparing a pigment using microorganisms comprising:
a seed culture step of culturing a strain of skin flora with the accession number KCTC15741BP (Korea Research Institute of Bioscience and Biotechnology, Biological Resource Center) to prepare a seed culture;
a pre-culture step of culturing the seed culture in a medium containing vegetable oil to prepare a pre-culture;
a main fermentation step of adding the pre-culture to the medium containing vegetable oil to prepare a fermented product;
a step of separating the precipitate from the fermented product; and
a step of extracting a pigment from the precipitate.

According to one embodiment, the medium in the seed culture step may contain casein, soybean, dextrose, sodium chloride and dipotassium phosphate. Specifically, the medium in the seed culture step may contain 5 to 30 g/L of casein, 1 to 10 g/L of soybean, 1 to 6 g/L of dextrose, 1 to 10 g/L of sodium chloride, and 1 to 6 g/L of dipotassium phosphate.

According to one embodiment, the medium in the pre-culture step may contain soybean, yeast extract, glycerin, potassium phosphate, dipotassium phosphate and vegetable oil. Specifically, the medium in the pre-culture step may contain 0.01 to 3 g/L of soybean, 0.1 to 5 g/L of yeast extract, 0.1 to 5 g/L of glycerin, 1 to 15 g/L of potassium phosphate, 1 to 10 g/L of dipotassium phosphate, and 10 to 100 ml/L of vegetable oil.

According to one embodiment, the medium in the main fermentation step may contain soybean, yeast extract, glycerin, potassium phosphate, dipotassium phosphate, ammonium sulfate, (magnesium sulfate, copper sulfate or iron sulfate), potassium nitrate, calcium chloride, amino acid and vegetable oil. Specifically, the medium in the main fermentation step may contain 0.01 to 3 g/L of soybean, 0.1 to 5 g/L of yeast extract, 0.01 to 5 g/L of glycerin, 1 to 10 g/L of potassium phosphate, 1 to 10 g/L of dipotassium phosphate, 0.1 to 5 g/L of ammonium sulfate, 0.01 to 5 g/L of (magnesium sulfate, copper sulfate or iron sulfate), 0.01 to 3 g/L of potassium nitrate, 0.01 to 3 g/L of calcium chloride, 0.005 to 0.5 g/L of amino acid and 10 to 800 ml/L of vegetable oil.

### According to One Embodiment,

the seed culture step may comprise culturing under aerobic conditions of 25 to 35° C.,
the pre-culture step may comprise introducing 10 to 500 g/L of the seed culture and culturing under aerobic conditions of 25 to 35° C., and
the main fermentation step may comprise introducing 10 to 500 g/L of the pre-culture and culturing under aerobic conditions of 25 to 35° C.

### According to One Embodiment, the Present Invention May Comprise:

a step of subculturing to the pre-culture medium when the absorbance at a wavelength of 600 nm is 0.05 to 0.2 in the seed culture step;
a step of subculturing to the main fermentation when the absorbance at a wavelength of 600 nm is 0.2 to 1 in the pre-culture step; or
a step of completing the main fermentation 90 to 130 hours after the start of the main fermentation.

According to one embodiment, the separating step may comprise centrifuging the fermented product to remove bacterial cells, and adjusting the pH and then allowing it to stand.

According to one embodiment, the step of extracting the pigment may comprise mixing the precipitate and the solvent and vortexing the mixture, and centrifuging to obtain a supernatant.

Specific details of other implementations according to the present invention are included in the detailed description below.

### Effect of the Invention

According to the present invention, there is provided a pigment derived from natural substances as an active ingredient having functions such as killing bacteria and healing wounds. The pigment of the present invention can be utilized in various modifications by using substitution of metal ions or functional groups.

### BRIEF DESCRIPTION OF THE DRAWINGS

FIG. 1 is a photograph visually observing the fermented product.

FIG. 2 is a photograph visually observing the precipitates.

FIG. 3 is a photograph visually observing the pigment extract.

FIG. 4 is a photograph visually observing the pigment extract under visible light and ultraviolet light.

FIGS. 5 and 6 are graphs showing ultraviolet/visible (UV/vis) spectra of pigment extracts.

FIG. 7 is a chromatogram showing the results of TLC analysis of the pigment extract.

FIG. 8 is a graph showing the results of Fourier Transform Infrared spectroscopy (FT-IR) analysis of the pigment extract.

FIG. 9 is a photograph showing the visual confirmation of the pigment extract from the precipitate according to the solvent.

FIGS. 10 and 11 are photographs visually observing pigment extracts according to solvent under visible light and ultraviolet light.

FIG. 12 is a graph showing the ultraviolet/visible (UV/vis) spectrum of FIG. 11.

FIG. 13 is a graph showing the results of cytotoxicity evaluation by MTT analysis.

### BEST MODE FOR CARRYING OUT THE INVENTION

The present invention can be modified in various ways and has many embodiments, and specific embodiments will be exemplified and described in detail. However, this is not intended to limit the present invention to specific embodiments, but should be understood to include all transformations, equivalents, or substitutes included in the spirit and technical scope of the present invention. In explaining the present invention, if it is determined that a detailed description of a related known technology may obscure the gist of the present invention, the detailed description is omitted.

Hereinafter, the pigment according to the present invention and the method for preparing the same will be described in detail.

The pigment of the present invention can be produced from microorganisms, for example, skin flora. For example, the present invention may utilize a strain of Epidermidibacterium keratini sp., specifically, a strain having the accession number KCTC15741BP (Korea Research Institute of Bioscience and Biotechnology, Biological Resource Center).

Skin flora can produce porphyrinoids when cultured and fermented under specific conditions. Porphyrinoids can exhibit strong antioxidant properties depending on their structure. In addition, porphyrinoids may be applied in the cosmetic field as active ingredients with photodynamic therapy functions that react to light to kill harmful bacteria or help heal wounds. Since metal ions or functional groups in the structure can be substituted, it can be transformed into various structures and utilized according to the function.

According to one aspect, the present invention provides a method for preparing a pigment using microorganisms, the method comprising a seed culture step of culturing a strain of skin flora with the accession number KCTC15741BP (Korea Research Institute of Bioscience and Biotechnology, Biological Resource Center) to prepare a seed culture; a pre-culture step of culturing the seed culture in a medium containing vegetable oil to prepare a pre-culture; a main fermentation step of adding the pre-culture to the medium containing vegetable oil to prepare a fermented product; a step of separating the precipitate from the fermented product; and a step of extracting a pigment from the precipitate.

According to one embodiment, the medium in the seed culture step may contain casein, soybean, dextrose, sodium chloride and dipotassium phosphate. Specifically, the medium in the seed culture step may contain 5 to 30 g/L, for example, 10 to 25 g/L, or 15 to 20 g/L of casein, 1 to 10 g/L, for example, 2 to 8 g/L, or 2 to 4 g/L of soybean, 1 to 6 g/L, for example, 1 to 4 g/L, or 2 to 3 g/L of dextrose, 1 to 10 g/L, for example 2 to 8 g/L, or 4 to 6 g/L of sodium chloride, and 1 to 6 g/L, for example 2 to 5 g/L, or 2 to 3 g/L of dipotassium phosphate.

According to one embodiment, the medium in the pre-culture step of the present invention may contain soybean, yeast extract, glycerin, potassium phosphate, dipotassium phosphate and vegetable oil. Specifically, the medium in the pre-culture step may contain 0.01 to 3 g/L, for example 0.05 to 2 g/L, or 0.05 to 1 g/L of soybean, 0.1 to 5 g/L, for example 0.1 to 3 g/L, or 0.5 to 2 g/L of yeast extract, 0.1 to 5 g/L, for example 0.1 to 3 g/L, or 0.5 to 2 g/L of glycerin, 1 to 15 g/L, for example 1 to 10 g/L, or 3 to 6 g/L of potassium phosphate, 1 to 10 g/L, for example 1 to 8 g/L, or 2 to 6 g/L of dipotassium phosphate, and 10 to 100 ml/L, for example 20 to 80 ml/L, or 30 to 60 ml/L of vegetable oil.

According to one embodiment, the medium in the main fermentation step may contain soybean, yeast extract, glycerin, potassium phosphate, dipotassium phosphate, ammonium sulfate, (magnesium sulfate, copper sulfate or iron sulfate), potassium nitrate, calcium chloride, amino acid and vegetable oil. Specifically, for example, the medium in the main fermentation step may contain 0.01 to 3 g/L, for example 0.05 to 2 g/L, or 0.05 or 1 g/L of soybean, 0.1 to 5 g/L, for example 0.1 to 3 g/L, or 0.5 to 2 g/L of yeast extract, 0.01 to 5 g/L, for example 0.05 to 3 g/L, or 0.05 to 2 g/L of glycerin, 1 to 10 g/L, for example 2 to 8 g/L, or 3 to 6 g/L of potassium phosphate, 1 to 10 g/L, for example 1 to 8 g/L, or 2 to 6 g/L of dipotassium phosphate, 0.1 to 5 g/L, for example 0.5 to 3 g/L, or 0.5 to 2 g/L of ammonium sulfate, 0.01 to 5 g/L, for example 0.05 to 3 g/L, or 0.05 to 1 g/L of (magnesium sulfate, copper sulfate or iron sulfate), 0.01 to 3 g/L, for example 0.05 to 2 g/L, or 0.05 to 1 g/L of potassium nitrate, 0.01 to 3 g/L, for example 0.05 to 2 g/L, or 0.1 to 1 g/L of calcium chloride, 0.005 to 0.5 g/L, for example 0.01 to 0.3 g/L, or 0.01 to 0.1 g/L of amino acid and 10 to 800 ml/L, for example 50 to 500 ml/L, or 50 to 300 ml/L, or 50 to 150 ml/L of vegetable oil.

According to one embodiment, the amino acid may include one or more of glycine, alanine, serine, threonine, cysteine, valine, leucine, isoleucine, methionine, proline, phenylalanine, tyrosine, tryptophane, aspartame, glutamate, asparagine, glutamine, histidine, lysine, and arginine.

According to the specific embodiment, the medium in the main fermentation step of the present invention may contain 0.1 g/L of soybean (papain digest soybean), 1.0 g/L of yeast extract, 1.0 g/L of glycerin, 4.5 g of monopotassium phosphate, 3.0 g/L of dipotassium phosphate, 1.0 g/L of ammonium sulfate, 0.5 g/L of magnesium sulfate heptahydrate, 0.1 g/L of potassium nitrate, 0.3 g/L of calcium chloride, 0.08 g/L of glutamate, 100 ml/L of olive oil, and 888.42 g/L of distilled water.

According to one embodiment, the seed culture step of the present invention may comprise culturing under aerobic conditions of 25 to 35° C., for example 28 to 32° C. The seed culture may be diluted in a sterilized solution of 0.85% sodium chloride to 10% (v/v) and the seed culture may be subcultured to the pre-culture when the absorbance is 0.05 to 0.2, for example, 0.05 to 0.15 as measured at a wavelength of 600 nm using a spectrophotometer.

According to one embodiment, the pre-culture step of the present invention may comprise adding 10 to 500 g/L, for example 50 to 300 g/L, or 50 to 200 g/L of the seed culture, and culturing under aerobic conditions at 25 to 35° C., for example 28 to 32° C. The pre-culture may be diluted in a sterilized solution of 0.85% sodium chloride to 10% (v/v) and the pre-culture may be subcultured to the main fermentation when the absorbance is 0.2 to 1, for example, 0.3 to 0.8 as measured at a wavelength of 600 nm using a spectrophotometer.

According to one embodiment, the main fermentation step of the present invention may comprise adding 10 to 500 g/L of pre-culture, for example 50 to 300 g/L, or 50 to 200 g/L, and culturing under aerobic conditions at 25 to 35° C., for example 28 to 32° C. The main fermentation step may comprise centrifuging 1 ml of the main fermentation solution at 13,000 rpm for 2 minutes to remove bacterial cells, diluting the supernatant with methanol to 30% (v/v), and then completing the main fermentation when the absorbance value is the highest as measured at a wavelength of 406 nm, such as 90 to 130 hours, or 100 to 120 hours, or 110 to 120 hours after the start of the main fermentation.

According to one embodiment, the step of separating the precipitate from the fermented product may comprise centrifuging the fermented product at 2000 to 10000 rpm, for example, 3000 to 8000 rpm, or 4000 to 6000 rpm for 10 to 40 minutes, for example, 10 to 30 minutes, or 15 to 25 minutes, to remove bacterial cells. Additionally, the separating step may comprise adjusting the pH to 1 to 3, and allowing it to stand overnight at 40 to 80° C., for example, 50 to 80° C., or 60 to 80° C. In addition, the separating step may be performed by repeatedly performing centrifugation, and the order and number of centrifugation processes are not limited.

According to one embodiment, the step of extracting the pigment from the precipitate may comprise mixing the precipitate and the solvent and vortexing the mixture. Specifically, the precipitate and the solvent may be mixed at a ratio of 1:10 to 1:40 (w/v), or 1:10 to 1:30 (w/v), or 1:15 to 1:25 (w/v), and vortexed for 10 seconds to 5 minutes, for example 30 seconds to 3 minutes, or 30 seconds to 2 minutes, or 30 seconds to 90 seconds. After vortexing, centrifugation may be performed at 7,000 to 20,000 rpm, for example 10,000 to 15,000 rpm, for 1 to 5 minutes, for example 1 to 3 minutes, to remove impurities, thereby obtaining a pigment extract in the supernatant. The solvent may include, for example, water, ethanol, methanol, butylene glycol, pentylene glycol, dipropylene glycol, propandiol, hexanediol, butanol, ethyl acetate, etc., and it is not particularly limited as long as it can be used as extraction solvents for cosmetics.

The vegetable oil of the present invention is not particularly limited as long as it is a vegetable oil without cytotoxicity. Specifically, the vegetable oil may include, but is not particularly limited to, one or more of edible or human-friendly oils such as macadamia oil, sunflower seed oil, grape seed oil, canola oil, rice germ oil, olive oil, soybean oil, argan oil, brown rice oil, perilla oil, sesame oil, almond oil, peanut oil, corn oil, red ginseng oil, avocado oil, macadamia oil, coconut oil, rosehip oil, vitamin tree seed oil, shea tree fruit oil, oil palm oil, bergamot fruit oil, camellia seed oil, safflower seed oil, apricot kernel oil, poppy seed oil, evening primrose seed oil, castor seed oil, green tea seed oil, meadowfoam seed oil, flax seed oil, and hemp seed oil.

According to one aspect, the present invention can provide a skin external agent or cosmetic composition containing the pigment. The present invention can be manufactured in any formulation that is commonly manufactured in the arts and is dermatologically applicable.

Dermatologically applicable means that the composition can have an effective effect that is relatively non-toxic and harmless to the subject and may include an external agent that can be applied to the skin, and that side effects resulting from the composition do not reduce the efficacy of the active ingredient, do not cause serious irritation to the subject, and do not damage the activity and properties of the active ingredients.

According to one embodiment, the skin external agent or cosmetic composition of the present invention may comprise ingredients commonly applied to the skin, such as stabilizers, solubilizers, vitamins, pigments, fragrances, adjuvants, and carriers.

The dermatologically applicable composition of the present invention may be formulated as, for example, a solution, a suspension, a milky solution, an emulsion, a paste, a gel, a pack, a cream, a lotion, a powder, a soap, a surfactant-containing cleanser, an oil, a powder foundation, an emulsion foundation, a wax foundation, a spray, and a hair cosmetic, but is not limited thereto. Specifically, it can be manufactured in the form of a skin lotion, a skin softener, a skin toner, an astringent, a lotion, a gel, a milk lotion, a moisture lotion, a nutrition lotion, a massage cream, a nutrition cream, a moisture cream, a hand cream, a foundation, an essence, an ampoule, a nutrition essence, a pack, a soap, a hair shampoo, a foot shampoo, a cleansing foam, a cleansing lotion, a cleansing cream, a body lotion, and a body cleanser.

Hereinafter, embodiments of the present invention will be described in detail so that a person skilled in the art can easily implement the present invention. However, the present invention may be implemented in various different forms and is not limited to the embodiments described herein.

### EXAMPLES AND COMPARATIVE EXAMPLES

### Seed Culture

When the medium temperature was 30° C., a microorganism with the accession number KCTC15741BP (Korea Research Institute of Bioscience and Biotechnology, Biological Resource Center) was inoculated and cultured under aerobic conditions to prepare a seed culture.

The medium composition is 17.0 g/L of casein (pancreatic digest of casein), 3.0 g/L of soybean (papain digest of soybean), 2.5 g/L of dextrose, 5.0 g of sodium chloride, 2.5 g/L of dipotassium phosphate, and 970 g/L of distilled water.

The seed culture was diluted in a sterilized solution of 0.85% sodium chloride to 10% (v/v) and, the seed culture was subcultured to the pre-culture when the absorbance is 0.1 as measured at a wavelength of 600 nm using a spectrophotometer.

### Pre-Culture

When the medium temperature was 30° C., 100 g/L of the seed culture was added and cultured under aerobic conditions to prepare a pre-culture.

The medium composition is 0.1 g/L of soybean (papain digest soybean), 1.0 g/L of yeast extract, 1.0 g/L of glycerin, 4.5 g of monopotassium phosphate, 3.0 g/L of dipotassium phosphate, 50 ml/L of olive oil, and 940.4 g/L of distilled water.

The pre-culture was diluted in a sterilized solution of 0.85% sodium chloride to 10% (v/v), and the pre-culture was subcultured to the main fermentation when the absorbance is 0.5 to 0.6 as measured at a wavelength of 600 nm using a spectrophotometer.

### Main Fermentation

When the medium temperature was 30° C., 100 g/L of the pre-culture was added and cultured under aerobic conditions to prepare a fermented product. The medium composition is as shown in Tables 1 to 5, and the end point of fermentation is as follows. First, 1 ml of the main fermentation solution was centrifuged at 13,000 rpm for 2 minutes to remove bacterial cells, and the supernatant was diluted with methanol to 30% (v/v), and then the main fermentation was terminated when the absorbance value was the highest, as measured at a 10 wavelength of 406 nm, i.e., after 116 hours of main fermentation.

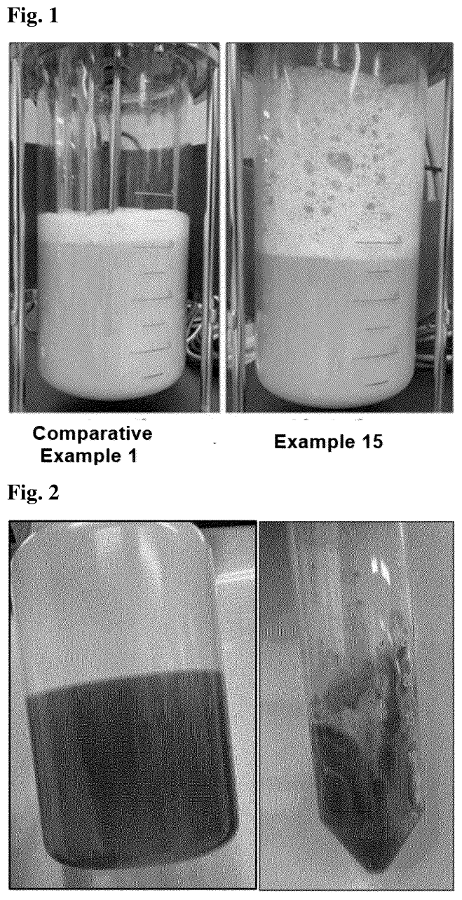

The state of the fermented product is shown in FIG. 1, and it is confirmed whether pigment was prepared.

TABLE 1

| Medium | Comparative | Exam- | Exam- | Exam- | Exam- | Exam- | Exam- | Exam- |
|---|---|---|---|---|---|---|---|---|
| composition | Example 1 | ple 1 | ple 2 | ple 3 | ple 4 | ple 5 | ple 6 | ple 7 |
| (w/v) | (g/L) | (g/L) | (g/L) | (g/L) | (g/L) | (g/L) | (g/L) | (g/L) |
| Papain Digest | 0.1 | 0.1 | 0.1 | 0.1 | 0.1 | 0.1 | 0.1 | 0.1 |
| of Soybean | | | | | | | | |
| Yeast extract | 1 | 1 | 1 | 1 | 1 | 1 | 1 | 1 |
| Glycerin | 1 | 1 | 1 | 1 | 1 | 1 | 1 | 1 |
| Monopotassium | 4.5 | 4.5 | 4.5 | 4.5 | 4.5 | 4.5 | 4.5 | 4.5 |
| Phosphate | | | | | | | | |
| Dipotassium | 3 | 3 | 3 | 3 | 3 | 3 | 3 | 3 |
| Phosphate | | | | | | | | |
| Ammonium | 1 | 1 | 1 | 1 | 1 | 1 | 1 | 1 |
| sulfate | | | | | | | | |
| Magnesium | 0.5 | 0.5 | 0.5 | 0.5 | 0.5 | 0.5 | 0.5 | 0.5 |
| sulfate | | | | | | | | |
| heptahydrate | | | | | | | | |
| Potassium | 0.1 | 0.1 | 0.1 | 0.1 | 0.1 | 0.1 | 0.1 | 0.1 |
| nitrate | | | | | | | | |
| Calcium | 0.3 | 0.3 | 0.3 | 0.3 | 0.3 | 0.3 | 0.3 | 0.3 |
| chloride | | | | | | | | |
| Glycine | — | 0.04 | — | — | — | — | — | — |
| Alanine | — | — | 0.05 | — | — | — | — | — |
| Serine | — | — | — | 0.06 | — | — | — | — |
| Threonine | — | — | — | — | 0.06 | — | — | — |
| Cysteine | — | — | — | — | — | 0.07 | — | — |
| Valine | — | — | — | — | — | — | 0.06 | — |
| Leucine | — | — | — | — | — | — | — | 0.07 |
| Olive oil | 100 | 100 | 100 | 100 | 100 | 100 | 100 | 100 |
| (ml/L) | | | | | | | | |
| Purified water | 888.5 | 888.46 | 888.45 | 888.44 | 888.44 | 888.43 | 888.44 | 888.43 |

TABLE 2

| Medium | Exam- | Exam- | Exam- | Exam- | Exam- | Exam- | Exam- | Exam- |
|---|---|---|---|---|---|---|---|---|
| composition | ple 8 | ple 9 | ple 10 | ple 11 | ple 12 | ple 13 | ple 14 | ple 15 |
| (w/v) | (g/L) | (g/L) | (g/L) | (g/L) | (g/L) | (g/L) | (g/L) | (g/L) |
| Papain Digest | 0.1 | 0.1 | 0.1 | 0.1 | 0.1 | 0.1 | 0.1 | 0.1 |
| of Soybean | | | | | | | | |
| Yeast extract | 1 | 1 | 1 | 1 | 1 | 1 | 1 | 1 |
| Glycerin | 1 | 1 | 1 | 1 | 1 | 1 | 1 | 1 |
| Monopotassium | 4.5 | 4.5 | 4.5 | 4.5 | 4.5 | 4.5 | 4.5 | 4.5 |
| Phosphate | | | | | | | | |
| Dipotassium | 3 | 3 | 3 | 3 | 3 | 3 | 3 | 3 |
| Phosphate | | | | | | | | |
| Ammonium | 1 | 1 | 1 | 1 | 1 | 1 | 1 | 1 |
| sulfate | | | | | | | | |
| Magnesium | 0.5 | 0.5 | 0.5 | — | 0.5 | 0.5 | 0.5 | 0.5 |
| sulfate | | | | | | | | |
| heptahydrate | | | | | | | | |
| Potassium | 0.1 | 0.1 | 0.1 | 0.1 | 0.1 | 0.1 | 0.1 | 0.1 |
| nitrate | | | | | | | | |
| Calcium | 0.3 | 0.3 | 0.3 | 0.3 | 0.3 | 0.3 | 0.3 | 0.3 |
| chloride | | | | | | | | |
| Isoleucine | 0.07 | — | — | — | — | — | — | — |
| Methionine | — | 0.08 | — | — | — | — | — | — |
| Proline | — | — | 0.06 | — | — | — | — | — |
| Phenylalanine | — | — | — | 0.09 | — | — | — | — |
| Tyrosine | — | — | — | — | 0.1 | — | — | — |
| Tryptophan | — | — | — | — | — | 0.11 | — | — |
| Aspartic Acid | — | — | — | — | — | — | 0.07 | — |
| Glutamate | — | — | — | — | — | — | — | 0.08 |
| Olive oil | 100 | 100 | 100 | 100 | 100 | 100 | 100 | 100 |
| (ml/L) | | | | | | | | |
| Purified water | 888.43 | 888.42 | 888.44 | 888.91 | 888.4 | 888.39 | 888.43 | 888.42 |

TABLE 3

| Medium | Exam- | Exam- | Exam- | Exam- | Exam- |
|---|---|---|---|---|---|
| composition | ple 16 | ple 17 | ple 18 | ple 19 | ple 20 |
| (w/v) | (g/L) | (g/L) | (g/L) | (g/L) | (g/L) |
| Papain Digest | 0.1 | 0.1 | 0.1 | 0.1 | 0.1 |
| of Soybean | | | | | |
| Yeast extract | 1 | 1 | 1 | 1 | 1 |
| Glycerin | 1 | 1 | 1 | 1 | 1 |
| Monopotassium | 4.5 | 4.5 | 4.5 | 4.5 | 4.5 |
| Phosphate | | | | | |
| Dipotassium | 3 | 3 | 3 | 3 | 3 |
| Phosphate | | | | | |
| Ammonium | 1 | 1 | 1 | 1 | 1 |
| sulfate | | | | | |
| Magnesium | 0.5 | 0.5 | 0.5 | 0.5 | 0.5 |
| sulfate | | | | | |
| heptahydrate | | | | | |
| Potassium | 0.1 | 0.1 | 0.1 | 0.1 | 0.1 |
| nitrate | | | | | |
| Calcium | 0.3 | 0.3 | 0.3 | 0.3 | 0.3 |
| chloride | | | | | |
| Asparagine | 0.07 | — | — | — | — |
| Glutamine | — | 0.08 | — | — | — |
| Histidine | — | — | 0.08 | — | — |
| Lysine | — | — | — | 0.08 | — |
| Arginine | — | — | — | — | 0.09 |
| Olive oil | 100 | 100 | 100 | 100 | 100 |
| (ml/L) | | | | | |
| Purified water | 888.43 | 888.42 | 888.42 | 888.42 | 888.41 |

TABLE 4

| Medium | Exam- | Exam- | Exam- | Exam- | Exam- | Exam- | Exam- | Exam- |
|---|---|---|---|---|---|---|---|---|
| composition | ple 21 | ple 22 | ple 23 | ple 24 | ple 25 | ple 26 | ple 27 | ple 28 |
| (w/v) | (g/L) | (g/L) | (g/L) | (g/L) | (g/L) | (g/L) | (g/L) | (g/L) |
| Papain Digest | 0.1 | 0.1 | 0.1 | 0.1 | 0.1 | 0.1 | 0.1 | 0.1 |
| of Soybean | | | | | | | | |
| Yeast extract | 1 | 1 | 1 | 1 | 1 | 1 | 1 | 1 |
| Glycerin | 1 | 1 | 1 | 1 | 1 | 1 | 1 | 1 |
| Monopotassium | 4.5 | 4.5 | 4.5 | 4.5 | 4.5 | 4.5 | 4.5 | 4.5 |
| Phosphate | | | | | | | | |
| Dipotassium | 3 | 3 | 3 | 3 | 3 | 3 | 3 | 3 |
| Phosphate | | | | | | | | |
| Ammonium | 1 | 1 | 1 | 1 | 1 | 1 | 1 | 1 |
| sulfate | | | | | | | | |
| Magnesium | 0.5 | 0.5 | 0.5 | 0.5 | 0.5 | 0.5 | 0.5 | 0.5 |
| sulfate | | | | | | | | |
| heptahydrate | | | | | | | | |
| Potassium | 0.1 | 0.1 | 0.1 | 0.1 | 0.1 | 0.1 | 0.1 | 0.1 |
| nitrate | | | | | | | | |
| Calcium | 0.3 | 0.3 | 0.3 | 0.3 | 0.3 | 0.3 | 0.3 | 0.3 |
| chloride | | | | | | | | |
| Glutamate | 0.08 | 0.08 | 0.08 | 0.08 | 0.08 | 0.08 | 0.08 | 0.08 |
| Rice germ oil | 100 | — | — | — | — | — | — | — |
| (ml/L) | | | | | | | | |
| Sunflower seed | — | 100 | — | — | — | — | — | — |
| oil (ml/L) | | | | | | | | |
| Grapeseed Oil | — | — | 100 | — | — | — | — | — |
| (ml/L) | | | | | | | | |
| Macadamia Oil | — | — | — | 100 | — | — | — | — |
| (ml/L) | | | | | | | | |
| Apricot kernel | — | — | — | — | 100 | — | — | — |
| oil (ml/L) | | | | | | | | |
| Canola Oil (ml/L) | — | — | — | — | — | 100 | — | — |
| Argan Oil (ml/L) | — | — | — | — | — | — | 100 | — |
| Camellia seed | — | — | — | — | — | — | — | 100 |
| oil (ml/L) | | | | | | | | |
| Purified water | 888.42 | 888.42 | 888.42 | 888.42 | 888.42 | 888.42 | 888.42 | 888.42 |

TABLE 5

| Medium | Example | Example | Example |
|---|---|---|---|
| composition | 29 | 30 | 31 |
| (w/v) | (g/L) | (g/L) | (g/L) |
| Papain Digest | 0.1 | 0.1 | 0.1 |
| of Soybean | | | |
| Yeast extract | 1 | 1 | 1 |
| Glycerin | 1 | 1 | 1 |
| Monopotassium | 4.5 | 4.5 | 4.5 |
| Phosphate | | | |
| Dipotassium | 3 | 3 | 3 |
| Phosphate | | | |
| Ammonium | 1 | 1 | 1 |
| sulfate | | | |
| Manganese(II) | 1 | — | — |
| sulfate | | | |
| pentahydrate | | | |
| Copper(II) | — | 1.04 | — |
| sulfate | | | |
| pentahydrate | | | |
| Iron(II) sulfate | — | — | 1.15 |
| heptahydrate | | | |
| Potassium | 0.1 | 0.1 | 0.1 |
| nitrate | | | |
| Calcium | 0.3 | 0.3 | 0.3 |
| chloride | | | |
| Glutamate | 0.08 | 0.08 | 0.08 |
| Olive oil (ml/L) | 100 | 100 | 100 |
| Purified water | 887.92 | 887.88 | 887.77 |

### Separation of Medium

The main fermented product obtained from Example 15 was centrifuged at 5,000 rpm for 20 minutes to remove bacterial cells, and 1 N HCl was added to adjust the pH to 2. After standing overnight at 70° C., centrifugation was performed at 5,000 rpm for 20 minutes to yield a dark red precipitate. The obtained precipitate is shown in FIG. 2.

### Extraction

The precipitate was mixed with methanol at a ratio of 1:20 (w/v) and vortexed vigorously for 1 minute. Afterwards, the solution was centrifuged at 13,000 rpm for 2 minutes to remove impurities, thereby obtaining a dark pink supernatant. The experimental examples below confirmed that the resulting pigment extract contains porphyrinoid-based substances.

The pigment extract obtained through the process is shown in FIG. 3.

### Experimental Example 1: Selection of Optimal Culture Condition

With improving the growth conditions of the strain with the accession number KCTC15741BP (Korea Research Institute of Bioscience and Biotechnology, Biological Resource Center), the phenomenon of porphyrinoid-based pigments being produced was observed. Therefore, experiments were conducted to explore the medium conditions under which the porphyrinoid-based pigments are most abundant.

The degree of pigment production was measured as follows. First, the fermented solution, which had undergone 96-hour main fermentation after the seed culture and pre-culture, was centrifuged (Hanil, Fleata40) at 3000 rpm for 15 minutes to remove bacterial cells. Afterwards, the supernatant was diluted with methanol to a final concentration of 30% (v/v). The absorbance of the diluted solution was measured at a wavelength of 406 nm using a spectrophotometer (BIO Teck, EPOCH2). The results are shown in Table 6.

TABLE 6

| | Comparative | Exam- | Exam- | Exam- | Exam- | Exam- | Exam- | Exam- |
|---|---|---|---|---|---|---|---|---|
| Item | Example 1 | ple 1 | ple 2 | ple 3 | ple 4 | ple 5 | ple 6 | ple 7 |
| A406 | —* | — | — | — | — | — | — | — |

| | Exam- | Exam- | Exam- | Exam- | Exam- | Exam- | Exam- | Exam- |
|---|---|---|---|---|---|---|---|---|
| Item | ple 8 | ple 9 | ple 10 | ple 11 | ple 12 | ple 13 | ple 14 | ple 15 |
| A406 | — | — | — | — | — | — | 0.425 | 1.081 |
| | Exam- | Exam- | Exam- | Exam- | Exam- | Exam- | Exam- | Exam- |
| Item | ple 16 | ple 17 | ple 18 | ple 19 | ple 20 | ple 21 | ple 22 | ple 23 |
| A406 | 0.166 | 0.273 | — | — | — | 0.737 | 0.834 | 0.476 |
| | Exam- | Exam- | Exam- | Exam- | Exam- | Exam- | Exam- | Exam- |
| Item | ple 24 | ple 25 | ple 26 | ple 27 | ple 28 | ple 29 | ple 30 | ple 31 |
| A406 | 0.912 | 0.555 | 0.594 | 0.719 | 0.646 | 0.857 | 0.771 | 0.693 |

*No absorbance

Comparing Comparative Example 1 with Examples 1 to 20, it is observed that the production of pigments varies depending on the type of amino acid. Among the 20 amino acids, Example 15 in which glutamate was added showed the highest absorbance as measured at a wavelength of 406 nm. These results appear to be related to the porphyrin biosynthetic pathway of Gram-positive bacteria.

From Examples 15 and 21 to 28, it is confirmed that olive oil used as a carbon source of the medium achieved the highest absorbance as measured at a wavelength of 406 nm.

In addition, from Example 15 and Examples 29 to 31, the effect of substituting the metal ion was observed. Among the four metal ions used in the experiment, magnesium sulfate heptahydrate achieved the highest absorbance as measured at a wavelength of 406 nm.

### Experimental Example 2: Ultraviolet (UV) Irradiation

Porphyrinoids have the characteristic of absorbing and emitting light with short wavelengths. At this time, strong fluorescence can be observed with the naked eyes.

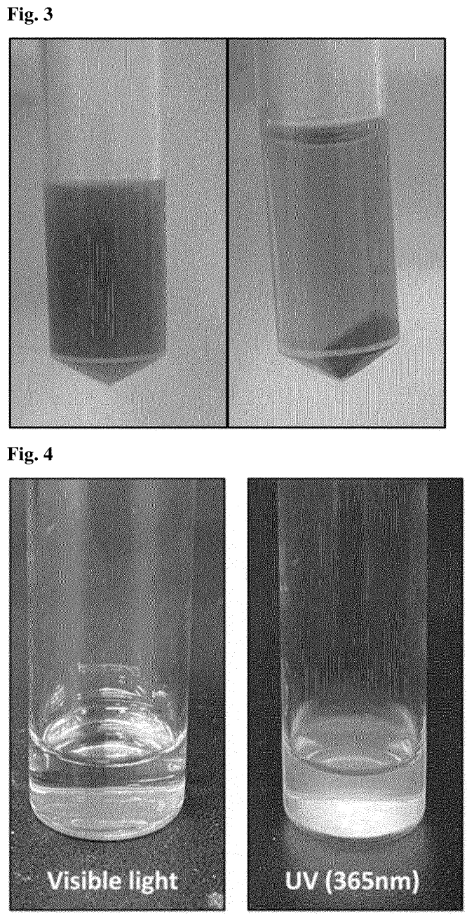

When the pigment extract of Example 15 was exposed to UV light with a wavelength of 365 nm, strong red fluorescence was observed. This is shown in FIG. 4.

### Experimental Example 3: UV/Vis Spectra

Porphyrinoids exhibit very specific UV/vis spectra patterns due to potential differences between molecules. While the wavelength at which the bands appear may vary depending on the type of free base porphyrin, whether metalation has occurred, and the functional group, one strong soret band and two or four weak Q bands are observed basically.

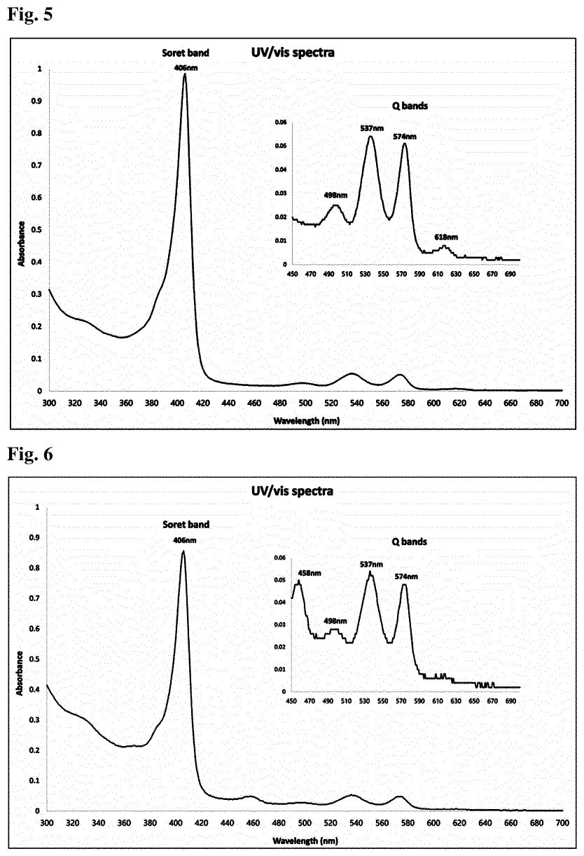

The UV/visible spectra measured from the pigment extract of Example 15 are shown in FIG. 5. Spectra were measured in the wavelength range of 200-700 nm using a spectrophotometer, and quartz cuvettes were used as a container for holding the sample. The measurement results showed one soret band at 406 nm. In addition, a total of four Q bands were observed at 498 nm, 537 nm, 574 nm, and 618 nm. It indicates that the substance contained in the extract of Example 15 is a porphyrinoid-based substance.

In addition, to examine the effect of the type of metal ion added to the medium on the pigment production of the strain having the accession number KCTC15741BP (Korea Research Institute of Bioscience and Biotechnology, Biological Resource Center), the UV/vis spectra measured for the extract of Example 29 are shown in FIG. 6. The measurement results showed one soret band at 406 nm. Additionally, a total of four Q bands were observed at 458 nm, 498 nm, 537 nm, and 574 nm. The UV/vis spectra of Examples 15 and 29 showed differences in the Q band. A shift in the Q band indicates a change in the potential of the porphyrinoid molecule. Since the potential of the molecule is affected by its structure and functional group, the porphyrinoids contained in the pigment extracts of Examples 15 and 29 are likely to have different structures, respectively.

Considering this, it is expected that by changing the type of metal ion included in the medium, porphyrinoid-based pigments having various structures can be obtained from the strain having the accession number KCTC15741BP (Korea Research Institute of Bioscience and Biotechnology, Biological Resource Center).

### Experimental Example 4: 1D Thin Layer Chromatography (1D TLC)

The pigment extracts of Example 15 were spotted onto a silica gel plate (TLC Silica gel 60 F254, Merck; 10 cm×5 cm) at the starting point (Base) and developed with a mobile phase containing methanol, ethyl acetate, and distilled water (3:5:1 v/v/v) to the end point (Front).

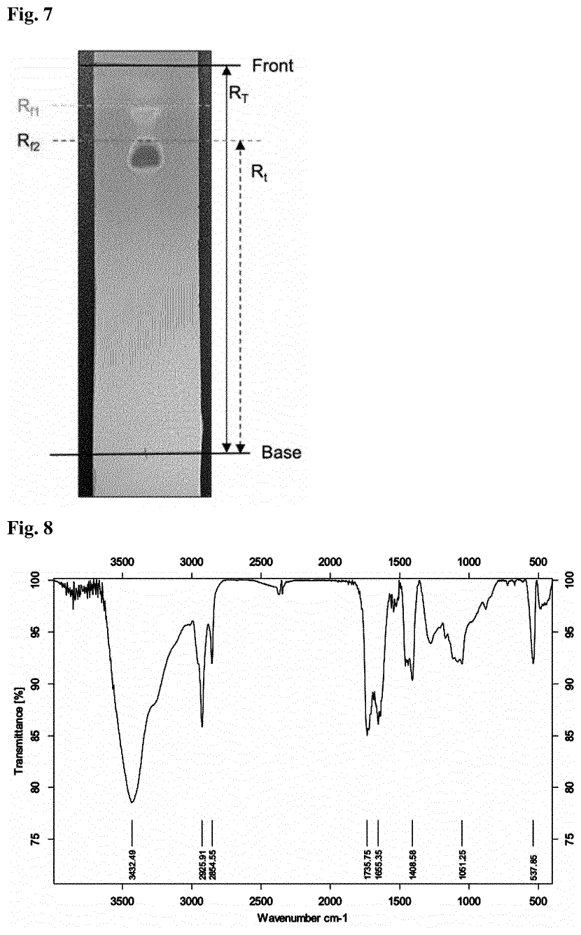

Color development was achieved by irradiating the naturally dried TLC plate with light of 365 nm wavelength. The results are shown in FIG. 7.

The results of 1D TLC showed that the pigment extract of Example 16 was a mixture containing at least two porphyrinoid-based substances. The Rf value of each spot was calculated using the equation below:

$${R}_{f} = {R}_{t} / {R}_{T}$$

(R_{T}: distance traveled by the mobile phase, Rₜ: distance traveled by the substance)
R_{f1} was calculated as 0.94 and R_{f2} as 0.83.

### Experimental Example 5: Fourier Transform Infrared Spectroscopy (FT-IR) Analysis

The spectrum generated by absorption of infrared rays can be used as data to obtain information about the chemical structure of a sample as well as qualitative and quantitative analysis of the substance.

The infrared absorption spectrum of the porphyrinoid extract of Example 15 was recorded in the infrared region of 4000 to 400 cm⁻¹ at a resolution of 0.4 cm⁻¹ or higher using an FT-IR spectrometer (Bruker, TENSOR27). The pretreatment for FT-IR analysis was to remove all the solvent from the porphyrinoid extract by evaporation, and then to apply remaining powders to the KBr pellet method. This is shown in FIG. 8.

When referring to tetraphenylporphyrin, which is the most basic structure of porphyrinoids, the bands that can appear are C═C, C—H, C═N, and N—H bands. C—H and N—H bands appear in the region of 1000-1500 cm⁻¹. The bands at 1501 cm⁻¹ and 1408 cm⁻¹ in FIG. 8 correspond to these. C═C and C═N bands appear in the region of 1500-2000 cm⁻¹. The bands at 1655 cm⁻¹ and 1735 cm⁻¹ in FIG. 8 correspond to these. Also, the bands appearing below 3000 cm⁻¹ among the bands around 3000 cm⁻¹, are the bands indicating alkyl C—H structures, to which the bands at 2854 cm⁻¹ and 2925 cm⁻¹ correspond. Finally, the N—H band appears as a broad band spanning 3000-3500 cm⁻¹. It can be seen that the band at 3432 cm⁻¹ in FIG. 8 indicates a N—H structure. It is presumed that the remaining bands may be attributed to tetraphenylporphyrin and other characteristic structures.

### Experimental Example 6: Application of Solvent for Cosmetics

To apply the porphyrinoid-based pigment produced by a strain having the accession number KCTC15741BP (Korea Research Institute of Bioscience and Biotechnology, Biological Resource Center) to the formulation, the precipitate isolated from the culture of Example 15 was extracted using a solvent applicable for cosmetics, and then diluted in 0.1 M phosphate buffer [pH 6.8].

First, an appropriate solvent was selected through preliminary experiments. The precipitate of Example 15 was divided into 0.05 g portions in 1 ml e-tubes and mixed with 1 ml of the solvent and vortexed vigorously for 1 minute. Then, centrifugation was performed at 13,000 rpm for 2 minutes to obtain the supernatant. The solvents used herein were a total of five types: 1,3-butyleneglycol (1,3-BG), pentyleneglycol (PG), dipropylene glycol (DPG), 1,3-propandiol (PDO), and 1,2-hexanediol (1,2-HXD). To visually observe whether extraction has occurred, methanol (MeOH) extract was used as a control group.

The results are shown in FIG. 9, and from the results of the experiment, it is found that pigments were extracted when pentylene glycol (PG) and 1,3-propanediol (PDO) were used.

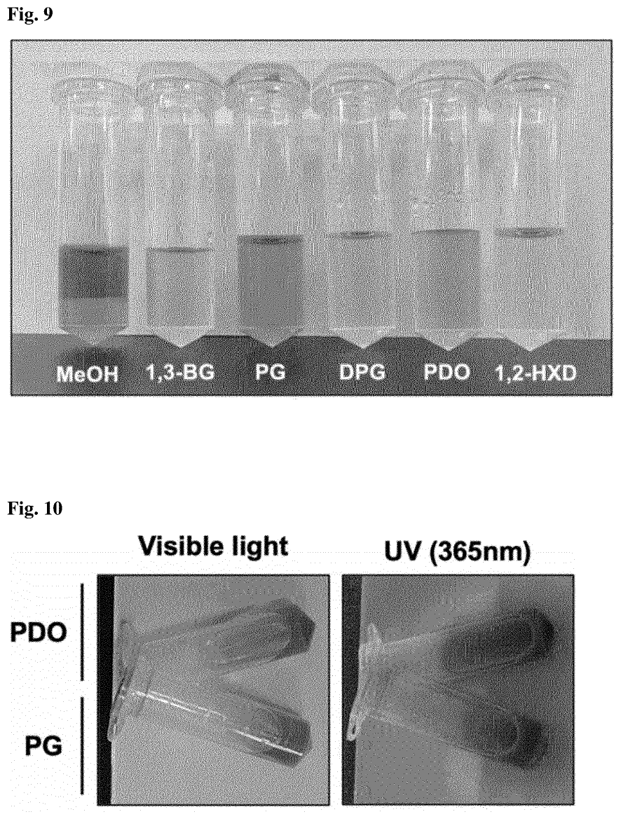

Next, the precipitate of Example 15 and the two types of solvents selected above were each added to a 100 ml baffle flask at 5% (w/v), and extraction was performed by stirring at 30° C. and 200 rpm for 1 hour. Then, centrifugation was performed at 13,000 rpm for 2 minutes to obtain the supernatant, and UV light at a wavelength of 365 nm was irradiated to visually confirm whether the porphyrinoid-based pigments were extracted. This is shown in FIG. 10.

Finally, each solvent extract of FIG. 10 was diluted in 0.1 M phosphate buffer [pH 6.8] to 1% (v/v) and the color development was observed under visible and UV rays. Additionally, UV/vis spectra were measured using the spectrophotometer to compare the values and patterns.

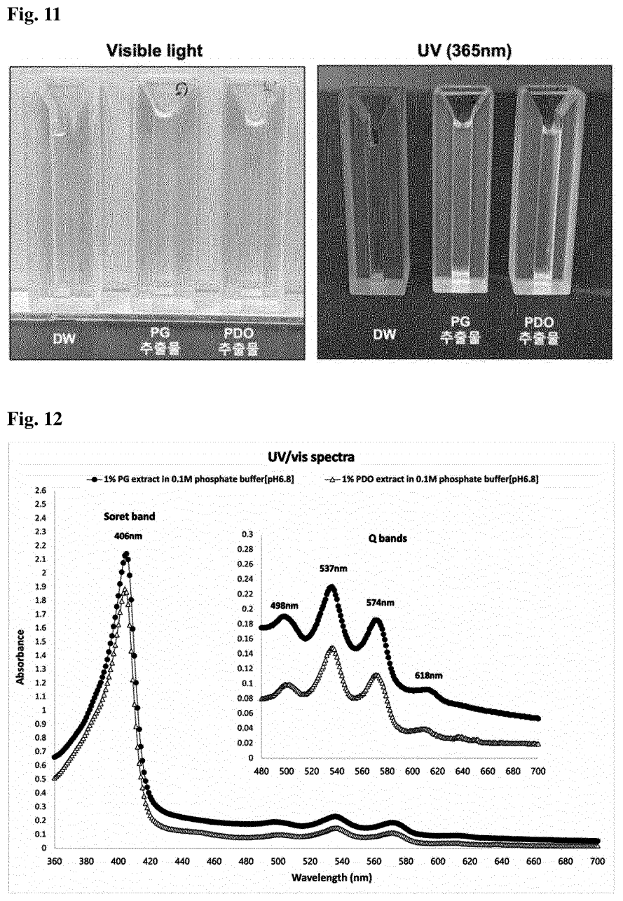

These are shown in FIGS. 11 and 12, respectively.

The precipitate of Example 15 was extracted with pentylene glycol (PG) and 1,3-propanediol (PDO), and both experimental groups were clear when mixed in a 0.1 M phosphate buffer solution. Additionally, they showed absorbances of 2.099 and 1.774 at a wavelength of 406 nm, respectively, with slightly higher value when extracted with PG. As the UV/vis spectrum pattern is same as in FIG. 5, it can be seen that the same component was extracted.

From Experimental Example 6, it is found that the porphyrinoid-based pigment prepared by the strain with the accession number KCTC15741BP (Korea Research Institute of Bioscience and Biotechnology, Biological Resource Center) can be extracted with solvents applicable to cosmetics, in addition to organic solvents, and thus is a water-soluble pigment applicable to water phase.

### Experimental Example 7: Cytotoxicity Test

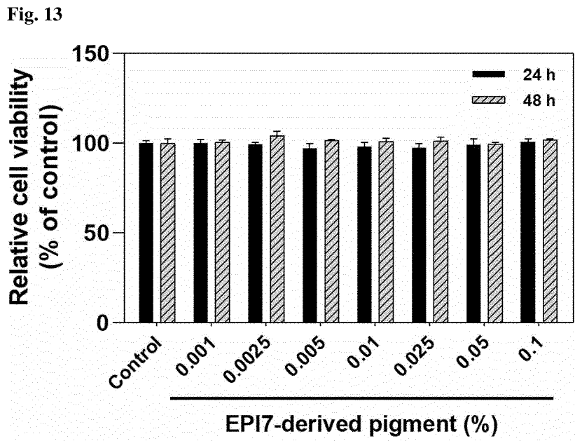

MTT test was performed to verify toxicity using cell viability as an indicator. HaCaT cells, which are human keratinocytes, were cultured in a 24-well culture plate at a concentration of 1×10⁵ cells/ml for 24 hours. After 2 hours of serum starvation, the cells were treated with various concentrations of porphyrinoid extracts and cultured for 24 and 48 hours. At this time, the porphyrinoid extract was extracted from the precipitate of Example 15 in DMSO at a concentration of 10% (v/w), and then diluted in a medium to 0.001, 0.0025, 0.005, 0.01, 0.025, 0.05, and 0.1%. To measure cell viability, all culture medium was removed, and cells were treated with MTT reagent and cultured for 2 hours. The resulting formazan was dissolved in DMSO and the absorbance was measured at 570 nm using a microplate reader, and the results are shown in FIG. 13.

As shown in FIG. 13, it is confirmed that the porphyrinoid extracts do not exhibit cytotoxicity.

As described above, the pigment of the present invention contains porphyrinoids that are produced by skin flora and are human-friendly and non-toxic. By substituting metal ions or functional groups in the molecular structure of porphyrinoids, it can have functional uses such as photodynamic therapy of killing bacteria or healing wounds, or antioxidant properties. Therefore, the pigment of the present invention has excellent functions as an external skin agent.

While specific embodiments of the present invention have been described in detail above, it should be understood by those skilled in the art that such specific description is merely a preferred embodiment, and the scope of the present invention is not limited to the specific embodiments described above.

1. A pigment prepared from a microorganism of skin flora with the accession number KCTC15741BP (Korea Research Institute of Bioscience and Biotechnology, Biological Resource Center).

2. The pigment prepared from a microorganism according to claim 1, wherein the pigment is a porphyrinoid-based substance.

3. A method for preparing a pigment using microorganisms, comprising:
a seed culture step of culturing a strain of skin flora with the accession number KCTC15741BP (Korea Research Institute of Bioscience and Biotechnology, Biological Resource Center) to prepare a seed culture;
a pre-culture step of culturing the seed culture in a medium containing vegetable oil to prepare a pre-culture;
a main fermentation step of adding the pre-culture to the medium containing vegetable oil to prepare a fermented product;
a step of separating the precipitate from the fermented product; and
a step of extracting a pigment from the precipitate.

4. The method for preparing a pigment using microorganisms according to claim 3, wherein the medium in the seed culture step contains casein, soybean, dextrose, sodium chloride and dipotassium phosphate.

5. The method for preparing a pigment using microorganisms according to claim 3, wherein the medium in the seed culture step contains 5 to 30 g/L of casein, 1 to 10 g/L of soybean, 1 to 6 g/L of dextrose, 1 to 10 g/L of sodium chloride, and 1 to 6 g/L of dipotassium phosphate.

6. The method for preparing a pigment using microorganisms according to claim 3, wherein the medium in the pre-culture step contains soybean, yeast extract, glycerin, potassium phosphate, dipotassium phosphate and vegetable oil.

7. The method for preparing a pigment using microorganisms according to claim 3, wherein the medium in the pre-culture step contains 0.01 to 3 g/L of soybean, 0.1 to 5 g/L of yeast extract, 0.1 to 5 g/L of glycerin, 1 to 15 g/L of potassium phosphate, 1 to 10 g/L of dipotassium phosphate, and 10 to 100 ml/L of vegetable oil.

8. The method for preparing a pigment using microorganisms according to claim 3, wherein the medium in the main fermentation step contains soybean, yeast extract, glycerin, potassium phosphate, dipotassium phosphate, ammonium sulfate, (magnesium sulfate, copper sulfate or iron sulfate), potassium nitrate, calcium chloride, amino acid and vegetable oil.

9. The method for preparing a pigment using microorganisms according to claim 3, wherein the medium in the main fermentation step contains 0.01 to 3 g/L of soybean, 0.1 to 5 g/L of yeast extract, 0.01 to 5 g/L of glycerin, 1 to 10 g/L of potassium phosphate, 1 to 10 g/L of dipotassium phosphate, 0.1 to 5 g/L of ammonium sulfate, 0.01 to 5 g/L of (magnesium sulfate, copper sulfate or iron sulfate), 0.01 to 3 g/L of potassium nitrate, 0.01 to 3 g/L of calcium chloride, 0.005 to 0.5 g/L of amino acid and 10 to 800 ml/L of vegetable oil.

10. The method for preparing a pigment using microorganisms according to claim 3, wherein:
the seed culture step comprises culturing under aerobic conditions of 25 to 35° C.,
the pre-culture step comprises introducing 10 to 500 g/L of the seed culture and culturing under aerobic conditions of 25 to 35° C., and
the main fermentation step comprises introducing 10 to 500 g/L of the pre-culture and culturing under aerobic conditions of 25 to 35° C.

11. The method for preparing a pigment using microorganisms according to claim 3, wherein the method comprises:
a step of subculturing to the pre-culture medium when the absorbance at a wavelength of 600 nm is 0.05 to 0.2 in the seed culture step;
a step of subculturing to the main fermentation when the absorbance at a wavelength of 600 nm is 0.2 to 1 in the pre-culture step; or
a step of completing the main fermentation 90 to 130 hours after the start of the main fermentation.

12. The method for preparing a pigment using microorganisms according to claim 3, wherein the separating step comprises:
centrifuging the fermented product to remove bacterial cells; and
adjusting the pH and then allowing it to stand.

13. The method for preparing a pigment using microorganisms according to claim 3, wherein the step of extracting the pigment comprises
mixing the precipitate and the solvent and vortexing the mixture; and
centrifuging to obtain a supernatant.
